SEQUENCE LISTING (20 sequences)

SEQ ID NO: 1 - protein, 54 aa, unidentified
  REGION 1..54: note = None
DGVKLCDVPSGTWSGHCGSSSKCSQQCKDREHFAYGGACHYQFPSVKCFCKRQC

SEQ ID NO: 2 - protein, 51 aa, unidentified
  REGION 1..51: note = None
QKLCQRPSGTWSGVCGNNNACKNQCIRLEKARHGSCNYVFPAHKCICYFPC

SEQ ID NO: 3 - protein, 50 aa, unidentified
  REGION 1..50: note = None
LCNERPSQTWSGNCGNTAHCDKQCQDWEKASHGACHKRENHWKCFCYFNC

SEQ ID NO: 4 - protein, 45 aa, unidentified
  REGION 1..45: note = None
RECKAQGRHGTCFRDANCVQVCEKQAGWSHGDCRAQFKCKCIFEC

SEQ ID NO: 5 - protein, 52 aa, unidentified
  REGION 1..52: note = None
MLCKLSMFGAVLGVPACAIDCLPMGKTGGSCEGGVCGCRKLTFKILWDKKFG

SEQ ID NO: 6 - protein, 38 aa, unidentified
  REGION 1..38: note = None
GFGCPLNQGACHNHCRSIKRRGGYCSGIIKQTCTCYRK

SEQ ID NO: 7 - protein, 41 aa, unidentified
  REGION 1..41: note = None
APGNKAECEREKGYCGFLKCSFPFVVSGKCSRFFFCCKNIW

SEQ ID NO: 8 - protein, 45 aa, unidentified
  REGION 1..45: note = None
FLPKTLRKFFCRIRGGRCAVLNCLGKEEQIGRCSNSGRKCCRKKK

SEQ ID NO: 9 - protein, 50 aa, unidentified
  REGION 1..50: note = None
RMCKTPSGKFKGYCVNNTNCKNVCRTEGFPTGSCDFHVAGRKCYCYKPCP

SEQ ID NO: 10 - protein, 41 aa, unidentified
  REGION 1..41: note = None
SPAIWGCDSFLGYCRLACFAHEASVGQKECAEGMLCCIPNV

SEQ ID NO: 11 - protein, 64 aa, unidentified
  REGION 1..64: note = None
ADIDFSTCARMDVPILKKAAQGLCITSCSMQNCGTGSCKKRSGRPTCVCYRCANGGGDIPLGAL

SEQ ID NO: 12 - protein, 53 aa, unidentified
  REGION 1..53: note = None
SCPGLCAKRSKTWSGWCGSSNNCDKQCRTKEGATHGACHGNILKRACDCYFKC

SEQ ID NO: 13 - protein, 45 aa, unidentified
  REGION 1..45: note = None
KTCENLADKYRGPCFSGCDTHCTTKENAVSGRCRDDFRCWCTKRC

SEQ ID NO: 14 - protein, 48 aa, unidentified
  REGION 1..48: note = None
RTCESQSHRFKGPCARKANCASVCNTEGFPDGYCHGVRRRCMCTKPCP

SEQ ID NO: 15 - protein, 36 aa, unidentified
  REGION 1..36: note = None
ACNFQSCWATCQAQHSIYFRRAFCDRSQCKCVFVRG

SEQ ID NO: 16 - protein, 30 aa, unidentified
  REGION 1..30: note = None
VGECVRGRCPSGMCCSQFGYCGKGPKYCGR

SEQ ID NO: 17 - protein, 45 aa, unidentified
  REGION 1..45: note = None
QQCGRQAGNRRCANNLCCSQYGYCGRTNEYCCTSQGCQSQCRRCG

SEQ ID NO: 18 - protein, 63 aa, unidentified
  REGION 1..63: note = None
GSNFCDSKCKLRCSKAGLADRCLKYCGICCEECKCVPSGTYGNKHECPCYRDKKNSKGKSKCP

SEQ ID NO: 19 - protein, 38 aa, unidentified
  REGION 1..38: note = None
AKCIKNGKGCREDQGPPFCCSGFCYRQVGWARGYCKNR

SEQ ID NO: 20 - protein, 46 aa, unidentified
  REGION 1..46: note = None
KSCCPNTTGRNIYNACRLTGAPRPTCAKLSGCKIISGSTCPSDYPK

(19) United States
(12) Patent Application Publication
(10) Pub. No.: US 2025/0319156 A1
(43) Pub. Date: Oct. 16, 2025

(54) Synergistic antimicrobial compositions containing selected peptides and fatty acids

(71) Applicant: CLEVER BIOSCIENCE S.R.L., Campospinoso Albaredo (PV) (IT)

(72) Inventors: Emma MAZZEI, Milano (IT); Elisa BREVIARIO, Bonate Sopra (BG) (IT); Stefano ZUCCHINALI, Pontirolo Nuovo (BG) (IT); Giorgio FRESCHI, Piacenza (IT)

(73) Assignee: CLEVER BIOSCIENCE S.R.L., Campospinoso Albaredo (PV) (IT)

(21) Appl. No.: 18/576,531

(22) PCT Filed: Jul. 14, 2022
(86) PCT No.: PCT/EP2022/069722

§ 371 Date: Jan. 4, 2024

(30) Foreign Application Priority Data
Jul. 14, 2021 (IT) 102021000018530

(51) Int. Cl. A61K 38/17; A01N 63/50; A01P 1/00; A01P 3/00; A61K 31/19; A61K 31/20; A61K 31/201

(52) U.S. Cl. CPC A61K 38/1729; A01N 63/50; A01P 1/00; A01P 3/00; A61K 31/19; A61K 31/20; A61K 31/201

(57) ABSTRACT

Novel synergistic compositions based on antimicrobial peptides and fatty acids are described. Antimicrobial peptides may be selected from the classes of defensins, thionins, heveins, snakins/GASA, knottins. Fatty acids may contain 4 to 22 carbon atoms and may be saturated, monounsaturated or polyunsaturated. The present peptides and fatty acids synergize, thereby providing a strong antifungal and antibacterial activity, with important applications, especially in the agronomic field.

### CROSS-REFERENCE TO RELATED APPLICATIONS

This application is a 371 of PCT/EP2022/069722, filed Jul. 14, 2022, which claims the benefit of Italian Patent Application No. 102021000018530, filed Jul. 14, 2021

### REFERENCE TO A SEQUENCE LISTING SUBMITTED ELECTRONICALLY VIA EFS-WEB

The content of the electronically submitted sequence listing, file name: 416-1018_SequenceListing.txt; size: 12 KB; and date of creation: Jan. 4, 2024, filed herewith, is incorporated herein by reference in its entirety.

### FIELD OF THE INVENTION

The present invention pertains the field of antimicrobial products and compositions containing the same, in particular for the use in the agronomic field.

### BACKGROUND OF THE INVENTION

Many plants have an advantageous, often essential, relationship with the microorganisms of the environment (water, soil, air). However, this relationship may become unbalanced: in this case, the microorganisms, in particular fungi and bacteria, transform into parasites and kill the plants by depriving them of nutritious substances. For example, fungal infections destroy every year more than 125 million tons of crops worldwide.

These contaminations are mainly dealt with antimicrobial synthetic products; however, the latter entail many problems: they kill also microorganisms that are beneficial for the soil, thereby causing serious consequences for plants. Moreover, the synthetic antimicrobial agents cause several environmental effects, in addition to toxicological problems for humans who come in contact with them. These problems are particularly relevant and urgent in the agronomic field, considering the wide surfaces to be treated and the corresponding massive use of antimicrobial agents, which is required to ensure sufficient product concentrations in the vicinity of all the plants to be treated. Further problems are those related to the appearance of resistant strains, with the consequent growing need of newer antimicrobial products with high efficacy.

In recent years, the regulations for environment protection have imposed limits to the use of synthetic antimicrobial agents, supporting at the same time the research of new products with natural characteristics, which should be less toxic for humans and easier to dispose of. The use of antimicrobial peptides, i.e. small protein molecules consisting of 10-100 amino acids, broadly occurring in nature (in bacteria, plants, insects, etc.) is very interesting. Currently, about 800 substances classified as antimicrobial peptides are known. The first to be studied were cecropins, isolated from silkworm (Hyalophora cecropia) in the early 1980s, and melittin, isolated from the venom of honeybee (Apis mellifera). The latter is one of the peptides studied most thoroughly and is therefore often used as a reference for studying new molecules. The skin of several amphibian species is a rich source of peptides (bombesins, magainins, temporins, etc.), produced and secreted by granular glands in response to a variety of stimuli. In humans and in other mammals (mouse, rat, rabbit), antimicrobial peptides belonging to the defensin family are stored in the form of granules in neutrophils (blood cells specialized in phagocytosis), whereas polymorphonuclear leukocytes of bovines are rich in peptides belonging to the cathelicidin family that showed in vitro and in vivo a significant antimicrobial activity.

Antimicrobial peptides have an action spectrum that is quite aspecific and thus generally broad against viruses, bacteria, fungi and protozoans; the activity arises rapidly and extends to microorganisms that have developed resistance. The action mechanism is attributable to the alteration of cellular membranes, with effects such as disorganization of membrane structure, alteration of permeability, outflow of cytoplasm components and cell lysis (destruction). Some peptides, such as buforin, directly interact with intracellular targets (DNA and/or RNA) inhibiting functions that are vital for the cell. Other peptides (for example those derived from cathelicidins and defensins) inhibit pro-inflammatory and immune-defense response of the host organism.

The low selectivity of these compounds on one hand widens the action spectrum; on the other hand, it involves a non-tailored interaction with the concerned microorganism, resulting in significant variations of potency across different microorganisms, making it difficult to maintain an average high level of activity against a large group of target microorganisms. A particularly resistant sub-group of microorganisms is that of phytoplasma, i.e. special bacteria without cell wall: they access the inner parts of the plant (phloema) via vector insects and cause serious diseases, even lethal for the plant; to date there are no curative strategies to contrast phytoplasma: in fact, the traditional antibacterial strategies, aimed to hit the bacterial cell wall, are ineffective on these microorganisms and the available treatments are limited to preventive ones.

The low specificity of antimicrobial peptides could also increase the risk of undesired effects on the plant and/or on humans. The possibility of reducing the amount/concentration of these products is not effectively viable since this is associated to an undesired reduction of treatment efficacy. Therefore, there is still the need for new antimicrobial products and compositions that associate the advantage of a broad action spectrum to the advantage of a stronger activity, such as to allow the use in amounts lower than the standard ones without compromising the extent of the effect. In particular, there is still the need for synergistic compositions that allow to obtain a higher antimicrobial effect than the sum of those obtainable by the components constituting the composition, taken separately. These problems have been addressed by the present inventors and unexpectedly solved in accordance with the following disclosures.

### SUMMARY

It has been now discovered that it is possible to obtain an unexpectedly high antimicrobial activity by combining antimicrobial peptides and fatty acids. In particular, antimicrobial peptides may be selected from the classes of defensins, thionins, heveins, snakins/GASA, knottins. Fatty acids may contain 4 to 22 carbon atoms and may be saturated, monounsaturated, or polyunsaturated. The present peptides and fatty acids synergize, thereby providing a strong antimicrobial, in particular antifungal and antibacterial, activity, with important applications, especially in the agronomic field. Selected combinations of fatty acids with antimicrobial peptides are also disclosed herein with additional advantages.

### DETAILED DESCRIPTION OF THE INVENTION

The compositions of the invention are characterized in that they comprise at least one antimicrobial peptide and at least one fatty acid as active ingredients. In said compositions, the above-mentioned ingredients are suitably formulated with excipients and a suitable carrier, in particular for the use in agriculture.

Any peptide with antimicrobial activity is suitable for being included in the present compositions. Antimicrobial peptides are per se widely known and described in the literature. In the present invention, peptides belonging to the classes of defensins, thionins, heveins, snakins/GASA, knottins proved to be very effective.

Defensins are a phylogenetically very old peptide family, with a highly conserved structure, that are present in mammals, insects and plants: they are amphipathic peptides capable of inserting in membranes and of inducing the pore formation resulting in death due to cells lysis. There are two main defensin categories: α and β and they differ in the type of producing cell and thus in localization. α defensins are mainly produced by neutrophils (contained in primary granules) and by Paneth cells; they are produced and secreted as an inactive form of pro-peptide and are activated by proteolytic cleavage by trypsin. β defensins are produced by epithelial cells of the respiratory system, the integumentary system, the urogenital system and the skin.

An interesting subclass of antimicrobial peptides is the one of plant defensins (Planta, 2002, 216, pp-193-202). Particularly interesting peptides among them are the following:
Hs-AFP1, corresponding to SEQ.ID.NO: 1
Rs-AFP2, corresponding to SEQ.ID.NO: 2
Ah-AMP1, corresponding to SEQ.ID.NO: 3
NmDef2, corresponding to SEQ.ID.NO: 4
Oh-DEF, corresponding to SEQ.ID.NO: 5
DefMT6, corresponding to SEQ.ID.NO: 6
AvBD1, corresponding to SEQ.ID.NO: 7
mDB14, corresponding to SEQ.ID.NO: 8
PsDef1, corresponding to SEQ.ID.NO: 9
Def-Tk, corresponding to SEQ.ID.NO: 10
Abf-2, corresponding to SEQ.ID.NO: 11
K7MPK0, corresponding to SEQ.ID.NO: 12
Def1.1, corresponding to SEQ.ID.NO: 13
OsDef8 corresponding to SEQ.ID.NO: 14
Termicin, corresponding to SEQ.ID.NO: 15

The peptides referred herein are per se known; for example, the peptide Hs-AFP1 is per se known from WO200472239, WO202186982 and WO2016205902; the peptide Rs-AFP2 is per se known e.g. from WO200109174 and WO200109175.

Another class of antimicrobial peptides that is particularly effective in the present invention is the class of heveins. They are peptides originating from the rubber tree (Hevea brasiliensis), that are obtained from the incision of the tree and have properties promoting latex coagulation. Heveins are the result of hydrolysis of the natural peptide (pro-hevein, containing 187 amino acids) into shorter fragments. Preferred examples of heveins that can be used in the present invention are the peptides:
Ay-AMP, corresponding to SEQ.ID.NO: 16
Ee-CBP, corresponding to SEQ.ID.NO: 17.

A further class of antimicrobial peptides that is particularly effective in the present invention is the class of snakins (also identified as GASA family). Snakins are plant antimicrobial peptides consisting of three distinct regions: an N-terminal signal peptide; a variable site; and the GASA domain in the C-terminal region composed of twelve cysteine residues that contribute to the biochemical stability of the molecule. These peptides are known to play different roles in response to a variety of stress factors. A preferred example of snakins that can be used in the present invention is the peptide:
StSN1, corresponding to SEQ.ID.NO: 18.

A further class of antimicrobial peptides that is particularly effective in the present invention is the class of knottins (cystine-knots (ICKs)). They are peptides characterized in that they contain three disulfide bridges, that form an intramolecular knot and give structural and functional resistance to high temperatures, to enzymatic degradation, to extreme pH and to mechanical stresses. The loops connecting the disulfide bridges show a high sequence variability, resulting in a wide range of functions. A preferred example of knottins that can be used in the present invention is the peptide:
McAMP1, corresponding to SEQ.ID.NO: 19.

Another class of antimicrobial peptides that is particularly effective in the present invention is the class of thionins. An important subclass thereof is the subclass of viscotoxins (Biophysical Journal Volume 85 August 2003 971-981). Among them, a peptide useful for the purposes of the invention is:
VtA3, corresponding to SEQ.ID.NO: 20.

A subgroup of peptide preferred according to the invention is the subgroup consisting of Hs-AFP1, Rs-AFP2 or PsDef-1, which have the above-mentioned structures.

The fatty acids that can be used in the present composition may be indifferently saturated, monounsaturated or polyunsaturated, being preferably selected in the interval C₄-C₂₂. Said fatty acids can be used as such and/or in the form of salts thereof and/or in the form of hydroxylated derivatives thereof; said variants are all included in the definition of “fatty acids” according to the present invention. Specific preferred fatty acids are: pelargonic acid, crotonic acid, caproleic acid. The fatty acids used in the invention can either have or not have antimicrobial activity per se: in any case they synergically interact with the peptide, thereby causing an overall antimicrobial activity higher than the sum of the activities of the two components taken separately.

A preferred sub-embodiment of the present invention is represented by new combinations of defensins with fatty acids selected from the group consisting of crotonic acid, pelargonic acid, caproleic acid and mixtures thereof. These combinations have shown a remarkably high level of synergic antimicrobial interaction (measured as FIC Index) against a large variety of target microorganisms, including fungi, Gram positive and Gram negative bacteria, inclusive of phytoplasma, thus conjugating the hardly conciliable effects of aspecificity and efficacy; the highest level of synergy is present when the defensins are combined with pelargonic acid, which represents an even more preferred combination. The obtained high levels of synergy are paralleled by a high level of antiinfective activity when applied on plant infections in open field, as confirmed by the experimental data included in this specification. A remarkable advantage of the combinations according to this sub-embodiment is their potent inhibitory activity against phytoplasma, a sub-class of bacteria responsible for hardly curable plant diseases, being resistant to conventional antibacterial agents; the level of activity is dramatically higher compared to that shown by the two separate products tested alone: this indicates that the synergism among these agents is especially amplified when the target microorganism is a phytoplasm; this is particularly unexpected because antimicrobial peptides and fatty acids are known to exert antimicrobial activity via interaction with the bacterial cell wall, a cellular component absent in phytoplasma. In the present sub-embodiment, any defensin can be used in combination with said crotonic, pelargonic and/or caproleic acid; examples of suitable defensins are: Hs-AFP1, corresponding to SEQ.ID.NO: 1; Rs-AFP2, corresponding to SEQ.ID.NO: 2; Ah-AMP1, corresponding to SEQ.ID.NO: 3; NmDef2, corresponding to SEQ.ID.NO: 4; Oh-DEF, corresponding to SEQ.ID.NO: 5; DefMT6, corresponding to SEQ.ID.NO: 6; AvBD1, corresponding to SEQ.ID.NO: 7; mDB14, corresponding to SEQ.ID.NO: 8; PsDef1, corresponding to SEQ.ID.NO: 9; Def-Tk, corresponding to SEQ.ID.NO: 10; Abf-2, corresponding to SEQ.ID.NO: 11; K7MPK0, corresponding to SEQ.ID.NO: 12; Def1.1, corresponding to SEQ.ID.NO: 13; OsDef8 corresponding to SEQ.ID.NO: 14; Termicin, corresponding to SEQ.ID.NO: 15. Particularly preferred are the combinations of crotonic acid, pelargonic acid and/or caproleic acid with one or more of said Hs-AFP1, Rs-AFP2 and PsDef1.

In all embodiments of the present invention, the peptides and the fatty acids can be combined with each other in all the possible proportions; preferably, neither of the two components is used in a weight ratio with respect to the other lower than 1:9. More preferably, the peptide (or their mixture, if more than one of them are used) is contained in a weight ratio with the fatty acid (or their mixture, if more than one of them are used) between 0.3:1 and 0.5:1; or alternatively between 0.5:1 and 1.5:1, for example in a 1:1 ratio.

In the present invention, the association of antimicrobial peptides with fatty acids obtains very high synergy levels, i.e. characterized by FIC index≤0.7, preferably between 0.05 and 0.5. According to the standard literature, the FIC Index can be calculated with the following formula:

$$FIC index = MICA / MICa + MICB / MICb$$

wherein “MICA and MICB” are the minimum inhibitory concentrations (MIC) of the two compounds A and B mixed with each other whereas “MICa and MICb” are the minimum inhibitory concentrations of the two components used singularly. FIC index<1.0 means synergy of the compounds combined with each other; FIC index=1.0 means that the compounds do not interact with each other; FIC index>1.0 means antagonism of the compounds combined with each other.

The term “antimicrobial” used herein is to be understood as comprising the terms antifungal, antibacterial, antiviral and antiparasitic. Preferably, the antimicrobial treatment is an antifungal or antibacterial treatment.

For the purposes of the antifungal treatment, all the fungal species can be treated according to the invention. Among them, the species preferably recommended for the purposes of the present treatment are the following.

In the agronomic field: Botrytis cinerea, Fusarium culmorum, Fusarium graminearum, Fusarium oxysporum, Fusarium solani, Stemphylium vesicarium, Scleratium rolfsii, Bipolaris sorokiniana, Sclerotinia sclerotiorum, Rhizoctonia solani, Zymoseptoria tritici, Cercospora beticola, Alternaria alternata, Venturia inequalis, Magnaporthe oryzae, Phytophtora infestans, Plasmopara viticola, Phakopsora pachyrhizi, Plasmopara viticola, Taphrina deformans, Uncinula necator, Erysiphe spp. Particularly preferred for the purposes of said treatment are the species: Botrytis cinerea, Fusarium culmorum, Fusarium graminearum, Phytophtora infestans, Alternaria alternata, Venturia inequalis.

In the pharmaceutical, nutraceutical or cosmetic field: Candida albicans, Aspergillus fumigatus, Cryptococcus neoformans, Malassezia furfur, Trichosporon spp, Histoplasma capsulatum, Blastomyces dermatitidis, Coccidioides immitis.

For the purposes of the antibacterial treatment, all the bacterial species, including phytoplasmas, can be treated according to the invention. Among them, the species preferably recommended for the purposes of the present treatment are the following.

In the agronomic field: Erwinia amylovora, Pseudomonas syringae, Xanthomonas campestris, Xanthomonas phaseoli, Xylella fastidiosa. Phytoplasmas that may be mentioned are for example: ‘Ca. Phytoplasma castaneae’, Ca. Phytoplasma graminis′, ‘Ca. Phytoplasma japonicum’, ‘Ca. Phytoplasma lycopersici’, ‘Ca. Phytoplasma oryzae’, ‘Ca. Phytoplasma pruni’, ‘Ca. Phytoplasma pyri’, ‘Ca. Phytoplasma solani’, ‘Ca. Phytoplasma vitis’.

In the pharmaceutical, nutraceutical or cosmetic field: Pseudomonas aeruginosa, Staphylococcus aureus, Escherichia coli, Campylobacter jejuni, Bacillus cereus, Listeria monocytogenes, Salmonella typhimurium, Clostridium perfringens. Preferably, bacterial species that can be treated are Escherichia coli, Staphylococcus aureus, Pseudomonas aeruginosa.

In the present compositions, said peptides and fatty acids are suitably formulated with a carrier suitable for dispersing the composition on a substrate that needs it, for example an aerial part of a plant that can be treated by superficial or endotherapic application or an agricultural land. Said composition, comprising the carrier, may be indifferently solid, semisolid, liquid, etc. Solid compositions may, for example, be in the form of powders, pellets, granules, microcapsules, etc.; said solid compositions may be delivered as such or may be previously dispersed in a liquid medium before administration on the land or on the plant. Semisolid compositions may be in the form of creams, pastes, gels, hydrogels, and the like. Liquid compositions may be in the form of a solution, suspension, dispersion, colloid, emulsion, etc.; they may be administered as such or in the form of an aerosol or spray. In an optional embodiment of the invention, the composition is not in the form of liposomes. Depending on their physical form, on the nature of the active ingredients contained, and on the usage conditions, the present compositions may include, in addition to the above-mentioned peptides, fatty acids and carrier, further excipients and other co-formulation agents according to the prior art of the field; in particular, they can contain stabilizers, antioxidants, buffering agents, chelating agents, agents for controlling the pH for example buffer systems, isotonicity agents, emulsifiers, co-emulsifiers, thickeners, gelling agents, film-forming agents, lubricants, glidants, anti-aggregating agents, moisture absorbers, coloring agents, etc.

Depending on their physical form and on the treatment needs (plant type and/or land type), the present compositions may be administered as such or dispersed in water, in fertilizing solutions, in biostimulating solutions, etc. For the purposes of an effective treatment, it is useful that the composition is administered in such an amount to provide a dose of mixture [peptide+fatty acid]/hectare (ha) of land between 50 Kg and 0.1 Kg, preferably between 5 Kg and 1 Kg.

A further object of the present invention is the use of a composition as defined above, comprising one or more antimicrobial peptides and one or more fatty acids, as an antimicrobial, preferably antifungal, agent. The present compositions can be used for both a preventive and a curative purpose, depending on the needs. The use is preferably intended in the agronomic field; however, the present association of peptides and fatty acids is also active in different fields and can be used without limitation for any antimicrobial treatment: said applications are equally part of the present invention. Therefore, the invention comprises also the preparation, provision and use of the present compositions in the pharmaceutical, nutraceutical or cosmetic field; the excipients and co-formulation agents used in these variants will be the one suitable for the respective pharmaceutical, nutraceutical, cosmetic use. A further object of the present invention is the use of one or more antimicrobial peptides and one or more fatty acids as defined above, in the preparation of an antimicrobial, preferably antifungal, composition.

A further object of the present invention is a process for the preparation of an antimicrobial composition with high synergistic activity, preferably for agronomic use, said process comprising formulating with each other: one or more antimicrobial peptides as defined above, one or more fatty acids as defined above, and, optionally, a suitable carrier and/or co-formulation agents.

The present invention is now described by way of the following non-limiting examples.

### EXPERIMENTAL PART

### Example 1—Evaluation of Synergism

### General Procedure

The antimicrobial activity was evaluated using the in-vitro susceptibility test with the microdilution method described in the Clinical and Laboratory Standard Institute protocols (M07—Methods for Dilution Antimicrobial Susceptibility Test for Bacteria That Grow Aerobically; M27—Reference Method for Broth Dilution Antifungal Susceptibility Testing of Yeasts; Reference Method for Broth Dilution Antifungal Susceptibility Testing of Filamentous Fungi). The MIC (Minimum Inhibitory Concentration) for each compound of interest was determined by these methods.

The positive control of the antimicrobial activity was carried out using fluconazole (for fungi and yeasts) and ceftriaxone (for bacterial strains). The negative control (absence of the active compounds) was evaluated by observing the correct microbial growth of the species of interest.

The synergistic activity of the various compounds was evaluated in vitro with the microdilution method in 96-well plates. Samples of the compounds of interest were prepared by diluting said compounds in sterile physiological solution to a specific concentration of 4 times the previously determined MIC. Then, combinations at different concentrations of the antimicrobial peptides with the fatty acids were made and said samples were treated as described in the CLSI protocols.

The synergy between the peptides and the fatty acids according to the invention was evaluated by calculating the FIC index according to the following formula:

$$FIC index = MICA / MICa + MICB / MICb$$

wherein “MICA and MICB” are the minimum inhibitory concentrations (MIC) of the two compounds A and B mixed with each other whereas “MICa and MICb” are the minimum inhibitory concentrations of the two components used singularly.

A value of FIC index<1.0 means synergy of the compounds combined with each other; a value of FIC index=1.0 means the absence of synergy of the compounds combined with each other; a value of FIC index>1.0 means antagonism of the compounds combined with each other.

The experimental results obtained are shown in Table I and in Table II:

TABLE I
Antifungal activity

| Product A | Product B | FIC | |
|---|---|---|---|
| (Peptide) | (Fatty acid) | INDEX | Microorganism |
| Hs-AFP1, | Pelargonic acid | 0.1-0.3 | Botrytis cinerea |
| Rs-AFP2 or | Crotonic acid | 0.5-0.7 | Fusarium culmorum |
| PsDef1 | Caproleic acid | 0.5-0.7 | Fusarium graminearum |
| | | | Bipolaris sorokiniana |
| | | | Sclerotinia sclerotiorum |
| | | | Rhizoctonia solani |
| | | | Zymoseptoria tritici |
| | | | Cercospora beticola |
| | | | Alternaria alternata |
| | | | Venturia inequalis |
| | | | Magnaporthe oryzae |
| | | | Phytophtora infestans |
| | | | Plasmopara viticola* |
| | | | Phakopsora pachyrhizi* |

TABLE II
Antibacterial activity

| Product A | Product B | FIC | |
|---|---|---|---|
| (Peptide) | (Fatty acid) | INDEX | Microorganism |
| Hs-AFP1, | Pelargonic acid | 0.1-0.3 | E.coli |
| Rs-AFP2 or | Crotonic acid | 0.4-0.7 | S.aureus |
| PsDef1 | Caproleic acid | 0.4-0.7 | P.aeruginosa |

### Example 2

Open Field Testing-Antifungal Activity on Fusarium graminearum by Mixtures of Fatty Acids and Peptides

The antifungal activity of three peptides (SEQ.ID.NOs.: 1, 2 and 9), of the crotonic and pelargonic acid and of the mixtures of these fatty acids with the aforementioned peptides was evaluated on winter wheat and durum wheat suitably contaminated by Fusarium graminearum. The peptides of SEQ.ID.NOs.: 1, 2 and 9 were dissolved in water at a concentration of 10% w/w. Aqueous solutions of crotonic and pelargonic acids were prepared at a concentration of 10% w/w. Aqueous solutions of peptides and solutions of fatty acids were mixed, in order to obtain six different mixtures at a concentration of 10% w/w of peptide and acid. The solutions were used at the dosages indicated in the table on both cultivars, after 2 days from the inoculation of the pathogenic strain Fusarium graminearum. Only one application was carried out at the time corresponding to the phenological scale BBCH (Biologische Bundesanstalt, Bundessortenamt and CHemical industry) 69-70 and the efficacy of the products was evaluated after 7 days for 3 weeks starting from the last check. The efficacy was assessed as incidence of leaves affected by the target pathogen compared to the untreated control: an increased % efficacy corresponds to a decreased number of leaves infested with the phytopathogen.

| | | Efficacy (%) | | |
|---|---|---|---|---|
| Sample | Concentration | 1° check | 2° check | 3° check |
| Hs_AFP1 | 2 L/ha | 35 | 27.4 | 26.5 |
| Rs_AFP2 | 2 L/ha | 38 | 34 | 25.4 |
| Ps_Def1 | 2 L/ha | 30 | 27 | 32 |
| Pelargonic acid | 2 L/ha | 10 | 8 | 9 |
| Crotonic acid | 2 L/ha | 8 | 7 | 10 |
| Hs_AFP1 + Pelargonic acid | 1 L/ha + 1 L/ha | 84 | 82 | 78 |
| Rs_AFP2 + Pelargonic acid | 1 L/ha + 1 L/ha | 78 | 75 | 72 |
| Ps_Def1 + Pelargonic acid | 1 L/ha + 1 L/ha | 78 | 72 | 73 |
| Hs_AFP1 + Crotonic acid | 1 L/ha + 1 L/ha | 80 | 77 | 75 |
| Rs_AFP2 + Crotonic acid | 1 L/ha + 1 L/ha | 76 | 73 | 69 |
| Ps_Def1 + Crotonic acid | 1 L/ha + 1 L/ha | 73 | 67 | 64 |
| Reference commercial product | 1 L/ha | 88 | 84 | 73 |
| Untreated | 0 | 0 | 0 | 0 |

### Example 3

Open Filed Testing on Phytoplasma-Antibacterial Activity on Ca. Phytoplasma vitis by Mixtures of Fatty Acids and Peptides

The activity on phytoplasma of three peptides (SEQ.ID.NO.: 1, 2 and 9), of crotonic and pelargonic acid and of the mixtures of these fatty acids with the aforementioned peptides were evaluated on Pervinca rosea suitably contaminated by Ca. Phytoplasma vitis.

The peptides of SEQ.ID.NO.: 1, 2 and 9 were dissolved in water at a concentration of 10% w/w. Aqueous solutions of crotonic and pelargonic acids were prepared at a concentration of 10% w/w. Aqueous solutions of peptides and solutions of fatty acids were mixed, in order to obtain six different mixtures at a concentration of 10% w/w of peptide and acid.

The evaluation of the efficacy of the products used was evaluated with ddPCR (Droplet Digital PCR) technique for the quantification of the genetic material (DNA and RNA) after 48 hours from exposure to endotherapy treatment. High % DNA and RNA values are related to reduced/no antibacterial activity. The solutions were used through endotherapic treatment at the dosages indicated in the following table:

| | | Efficacy (%) | |
|---|---|---|---|
| Sample | Concentration | % DNA | % RNA |
| Hs_AFP1 | 2 L/ha | 75 | 78 |
| Rs_AFP2 | 2 L/ha | 81 | 80 |
| Ps_Def1 | 2 L/ha | 82 | 80 |
| Crotonic acid | 2 L/ha | 76 | 84 |
| Pelargonic acid | 2 L/ha | 84 | 86 |
| Hs_AFP1 + crotonic acid | 1 L/ha + 1 L/ha | 42 | 48 |
| Rs_AFP2 + crotonic acid | 1 L/ha + 1 L/ha | 46 | 54 |
| Ps_Def1 + crotonic acid | 1 L/ha + 1 L/ha | 44 | 52 |
| Hs_AFP1 + pelargonic acid | 1 L/ha + 1 L/ha | 45 | 50 |
| Rs_AFP2 + pelargonic acid | 1 L/ha + 1 L/ha | 50 | 60 |
| Ps_Def1 + pelargonic acid | 1 L/ha + 1 L/ha | 52 | 52 |
| Untreated | 0 | 91 | 94 |

SEQUENCE LISTING
(Hs-AFP1)
SEQ.ID.NO: 1
DGVKLCDVPSGTWSGHCGSSSKCSQQCKDREHFAYGGACHYQFPSVKCFC
KRQC
(Rs-AFP2)
SEQ.ID.NO: 2
QKLCQRPSGTWSGVCGNNNACKNQCIRLEKARHGSCNYVFPAHKCICYFP
C
(Ah-AMP1)
SEQ.ID.NO: 3
LCNERPSQTWSGNCGNTAHCDKQCQDWEKASHGACHKRENHWKCFCYFNC
(NmDef2)
SEQ.ID.NO: 4
RECKAQGRHGTCFRDANCVQVCEKQAGWSHGDCRAQFKCKCIFEC
(Oh-DEF)
SEQ.ID.NO: 5
MLCKLSMFGAVLGVPACAIDCLPMGKTGGSCEGGVCGCRKLTFKILWDKK
FG
(DefMT6);
SEQ.ID.NO: 6
GFGCPLNQGACHNHCRSIKRRGGYCSGIIKQTCTCYRK
(AvBD1)
SEQ.ID.NO: 7
APGNKAECEREKGYCGFLKCSFPFVVSGKCSRFFFCCKNIW
(mDB14);
SEQ.ID.NO: 8
FLPKTLRKFFCRIRGGRCAVLNCLGKEEQIGRCSNSGRKCCRKKK
(PsDef1);
SEQ.ID.NO: 9
RMCKTPSGKFKGYCVNNTNCKNVCRTEGFPTGSCDFHVAGRKCYCYKPCP
(Def-Tk);
SEQ.ID.NO: 10
SPAIWGCDSFLGYCRLACFAHEASVGQKECAEGMLCCIPNV
(Abf-2);
SEQ.ID.NO: 11
ADIDFSTCARMDVPILKKAAQGLCITSCSMQNCGTGSCKKRSGRPTCVCY
RCANGGGDIPLGAL
(K7MPK0)
SEQ.ID.NO: 12
SCPGLCAKRSKTWSGWCGSSNNCDKQCRTKEGATHGACHGNILKRACDC
YFKC
(Def1.1);
SEQ.ID.NO: 13
KTCENLADKYRGPCFSGCDTHCTTKENAVSGRCRDDFRCWCTKRC
(OsDef8);
SEQ.ID.NO: 14
RTCESQSHRFKGPCARKANCASVCNTEGFPDGYCHGVRRRCMCTKPCP
(Termicin)
SEQ.ID.NO: 15
ACNFQSCWATCQAQHSIYFRRAFCDRSQCKCVFVRG
(Ay-AMP)
SEQ.ID.NO: 16
VGECVRGRCPSGMCCSQFGYCGKGPKYCGR
(Ee-CBP)
SEQ.ID.NO: 17
QQCGRQAGNRRCANNLCCSQYGYCGRTNEYCCTSQGCQSQCRRCG
(StSN1)
SEQ.ID.NO: 18
GSNFCDSKCKLRCSKAGLADRCLKYCGICCEECKCVPSGTYGNKHECPCY
RDKKNSKGKSKCP
(McAMP1)
SEQ.ID.NO: 19
AKCIKNGKGCREDQGPPFCCSGFCYRQVGWARGYCKNR
(VtA3)
SEQ.ID.NO: 20
KSCCPNTTGRNIYNACRLTGAPRPTCAKLSGCKIISGSTCPSDYPK

1.-15. (canceled)

16. A composition comprising an antimicrobial peptide selected from the class of defensins and a fatty acid selected from the group consisting of pelargonic acid, crotonic acid, caproleic acid, and mixtures thereof.

17. The composition of claim 16, wherein said defensins are selected from the group consisting of: Hs-AFP1, corresponding to SEQ.ID.NO: 1; Rs-AFP2, corresponding to SEQ.ID.NO: 2; Ah-AMP1, corresponding to SEQ.ID.NO: 3; NmDef2, corresponding to SEQ.ID.NO: 4; Oh-DEF, corresponding to SEQ.ID.NO: 5; DefMT6, corresponding to SEQ.ID.NO: 6; AvBD1, corresponding to SEQ.ID.NO: 7; mDB14, corresponding to SEQ.ID.NO: 8; PsDef1, corresponding to SEQ.ID.NO: 9; Def-Tk, corresponding to SEQ.ID.NO: 10; Abf-2, corresponding to SEQ.ID.NO: 11; K7MPK0, corresponding to SEQ.ID.NO: 12; Def1.1, corresponding to SEQ.ID.NO: 13; OsDef8 corresponding to SEQ.ID.NO: 14; and Termicin, corresponding to SEQ.ID.NO: 15.

18. The composition of claim 16, wherein the weight ratio of antimicrobial peptide to fatty acid is less than 1:9.

19. The composition of claim 18, wherein the weight ratio of antimicrobial peptide to fatty acid is between 0.3:1 and 0.5:1.

20. The composition of claim 18, wherein the weight ratio of antimicrobial peptide to fatty acid is between 0.5:1 and 1.5:1.

21. The composition of claim 18, wherein the weight ratio of antimicrobial peptide to fatty acid is 1:1.

22. The composition of claim 16, wherein the combination of the antimicrobial peptide and fatty acid have an FIC index of ≤0.7.

23. The composition of claim 22, wherein the FIC index is between 0.05 and 0.5.

24. A method of antimicrobial treatment, in the therapeutic or agronomic field, comprising administering the composition of claim 16 to a human or other mammal in need of such therapeutic treatment or to land or plants in need of such agronomic treatment.

25. The method of claim 24, wherein the antimicrobial treatment is for treating or preventing contaminations by fungi and/or bacteria, inclusive phytoplasma.

26. The method of claim 25, wherein said fungi are selected from the group consisting of Botrytis cinerea, Fusarium culmorum, Fusarium graminearum, Fusarium oxysporum, Fusarium solani, Stemphylium vesicarium, Scleratium rolfsii, Bipolaris sorokiniana, Sclerotinia sclerotiorum, Rhizoctonia solani, Zymoseptoria tritici, Cercospora beticola, Alternaria alternata, Venturia inequalis, Magnaporthe oryzae, Phytophtora infestans, Plasmopara viticola, Phakopsora pachyrhizi, Plasmopara viticola, Taphrina deformans, Uncinula necator, Erysiphe spp., Candida albicans, Aspergillus fumigatus, Cryptococcus neoformans, Malassezia furfur, Trichosporon spp, Histoplasma capsulatum, Blastomyces dermatitidis, and Coccidioides immitis.

27. The method of claim 25, wherein said bacteria are selected from the group consisting of Erwinia amylovora, Pseudomonas syringae, Xanthomonas campestris, Xanthomonas phaseoli, Xylella fastidiosa, Pseudomonas aeruginosa, Staphylococcus aureus, Escherichia coli, Campylobacter jejuni, Bacillus cereus, Listeria monocytogenes, Salmonella typhimurium, Clostridium perfringens, or phytoplasmas ‘Ca. Phytoplasma castaneae’, Ca. Phytoplasma graminis′, ‘Ca. Phytoplasma japonicum’, ‘Ca. Phytoplasma lycopersici’, ‘Ca. Phytoplasma oryzae’, ‘Ca. Phytoplasma pruni’, ‘Ca. Phytoplasma pyri’, ‘Ca. Phytoplasma solani’, and ‘Ca. Phytoplasma vitis’.

28. A process for the preparation of the composition of claim 16, comprising formulating with each other: an antimicrobial peptide selected from the class of defensins, and a fatty acid selected from the group consisting of pelargonic acid, crotonic acid, caproleic acid, and mixtures thereof and, optionally, a suitable carrier and/or suitable co-formulation agents.
